(19) United States
(12) Patent Application Publication
(10) Pub. No.: US 2004/0049245 A1
(43) Pub. Date: Mar. 11, 2004

(54) Autonomous patch for communication with an implantable device, and medical kit for using said patch

(76) Inventors: Volker Gass, Penthalaz (CH); Edward M. Gillis, Cupertino, CA (US)

Correspondence Address: FACTOR & PARTNERS, LLC, 1327 W. WASHINGTON BLVD., SUITE 5G/H, CHICAGO, IL, 60607, US

(21) Appl. No.: 10/238,014

(22) Filed: Sep. 9, 2002

(51) Int. Cl. A61N 1/08

(52) U.S. Cl. 607/60

(57) ABSTRACT

The present invention is directed to a self contained control patch for an implanted medical device, comprising a housing capable of being removably associated with an external surface of a patient, a transmitter for transmitting at least one of a transcutaneous power signal and a transcutaneous control signal to an implanted device; and a pre-programmed storage device containing at least one control program, wherein the control program dictates the nature of the signal from the transmitting means. Additionally, the present invention is directed to a system of such patches comprising two or more patches, with each patch being pre-programmed with a discrete set of instructions. Methods for using the patches are additionally disclosed.

### BACKGROUND OF THE INVENTION

1. Field of the Invention

The invention is directed generally to apparatuses for the control and power of implantable medical devices, and specifically to devices that utilize transcutaneous control and power transmission techniques.

2. Related Prior Art

Implantable medical devices have been utilized in the medical industry for years to provide constantly present, continually operable medical devices that enable medical treatment without constant medical supervision. These devices include neurostimulators, defibrillators, pacemakers, cochlear implants, and implantable pharmaceutical pumps, among others. The advent of these devices allowed for optimum patient mobility and independence, while still allowing for a high level of medical care.

One major issue with implantable medical devices is the invasive nature of the implantation process for the devices. In order to implant a device, generally a major surgical procedure must be undertaken, with all of the risks and recoveries that accompany that surgery. Further, once implanted, these devices can cause discomfort and cosmetic difficulties depending upon their size and configuration.

Thus, where implantable devices are used, it is desirable to minimize the size and nature of the device, while maximizing the autonomy of that device. Generally, an implantable medical device consists of a functional apparatus, and a power core to power the apparatus. Pacemakers, neurostimulators, cochlear implants, and defibrillators all provide electrical stimulation to different areas of the body in response to internal or external conditions. Pharmaceutical pumps provide force to deliver a drug to the body at a specified rate.

A number of approaches have been used to address the need for the combined autonomy and size of implantable devices. In order to provide for the power needs of the devices, while achieving some of the goals discussed above, significant research has gone into reducing the size of batteries that can be incorporated into the devices, as well as reducing the power consumption of the devices themselves.

One approach to reducing the size and configuration of implantable devices has been to remove the power source from the implanted device altogether, and instead transmit the power to the internal device using an externally located power source. Such conventional devices are well known in the art, and are disclosed, for example, in PCT WO 01/12108, and U.S. Pat. No. 5,814,089. Generally, these systems require the user to wear a portable power pack on, for example, a belt, which is then connected via electrical wiring to a transmitter located proximate the implanted device. The transmitter then transmits a signal to the internal device, generally via a radio frequency, powering the device.

The conventional system has a number of drawbacks, however. The portable power packs can be cumbersome and uncomfortable for a user to wear, and the required wiring can prove difficult to maneuver around. Additionally, the portable power packs themselves require a power source, which generally comes in a rechargeable format. Therefore, in order to maintain a continuous power supply to the implanted device, the power pack must continuously be provided with a freshly charged rechargeable power source. Such a system has provided difficulties in use in every day life.

Another improvement to implantable devices was drawn to minimizing the need for invasive surgical procedures during their operative life. Conventional implantable devices have generally included a pre-programmed set of instructions within the implanted device itself, called resident instructions, that direct operation of the device. In some cases, these instructions require reprogramming to alter the operation of the device as needed. For example, the specific delivery rates and timing instructions of an implantable pump may require reprogramming depending upon the stage of treatment the patient is in. Originally, conventional devices required surgery every time the specific operation parameters of the device needed to be altered. Obviously, the need for frequent surgical procedures is undesirable due to the invasiveness, and health issues of such procedures. Therefore, devices and methods were developed to transmit these instructions transdermally, allowing the devices to be reprogrammed without any surgery.

Conventional reprogramming devices are comprised of a computer and a reprogramming “wand” associated with the computer, as can be seen in U.S. Pat. No. 6,201,993. The computer is first accessed to select the particular programming instructions to be transmitted to the wand, and then the wand is placed proximate the area of the patient in which the device is implanted. The wand then transmits the new programming instructions to the device, generally via a conventional radio signal, reprogramming the device as needed.

Transdermal reprogramming opened up new areas of patient autonomy and functionality, allowing for brief doctor's visits to substitute for invasive and time consuming surgical procedures. The conventional reprogramming methods still left much to be desired, however. In these reprogramming methods, the patient is still required to report to a doctor's office to alter the treatment program. In some applications in which implanted devices are used, such as for providing pain medication, the waiting time for reprogramming is unacceptable. Additionally, since the conventional method requires a doctor's intervention in selecting the program for the wand to reprogram the implanted device, errors can occur, which can be harmful to the patient and impair a treatment regimen. Finally, on a practical note, the reprogramming apparatuses of the conventional computer and wand method can be extremely expensive, and can require long hours of training to learn to use.

Therefore, it is an object of the present invention to provide a simple, error free, and non-invasive device/system and method for the transmission of programming and power to an implanted device.

It is also an object of this invention to provide a system for using such a device to provide patient-specific programming abilities without the need for frequent doctor's visits.

It is a further object of this invention to provide a device/system having improved reliability of use, in both the short and long term.

These and other objections will become apparent to one of ordinary skill in the art in light of the present specification, claims and drawings appended hereto.

### SUMMARY OF THE INVENTION

The present invention is directed to a self-contained control patch for an implanted medical device, a system for using the patch, and a method for medical treatment comprising use of the patch system. In the preferred embodiment of the self-contained control patch, the patch comprises a housing capable of being removably associated with an external surface of a patient, means for transmitting at least one of a transcutaneous power signal and a transcutaneous control signal to an implanted device, and a pre-programmed storage device containing at least one control program, wherein the control program dictates the nature of the signal from the transmitting means. The transmitting means of the patch preferably comprises an RF transmitter, wherein the control program dictates at least one of the frequency and amplitude of the RF signal.

Alternatively, the transmitting means could comprise a conductive coil. In such an embodiment, the conductive coil is coupled with another conductive coil that is generally a part of an implanted device. The coils may then be used for the inductive transfer of power from the transmitting means to the implanted device, wherein the control program of the present device dictates the flux of the electric field through the coil in the implanted device.

In any of the previous embodiments, the transmitting means may transmit either power, or programming, or a combination of both.

The patch, as described, is preferably capable of being used in a pre-programmed treatment system. The system incorporates at least two self-contained control patches, each patch comprising a housing capable of being removably associated with an external surface of a patient, means for transmitting at least one of a transcutaneous power signal and a transcutaneous control signal to an implanted device, and a pre-programmed storage device containing at least one relatively fixed control program, wherein the control program dictates the nature of the signal from the transmitting means, wherein each control patch contains a separate generally fixed pre-programmed control program, and each control program may be individually implemented by the placement of the associated patch in operative position upon a patient. The patches used in this system are the same patches described above, with the same alternatives and embodiments.

The fixed programs associated with each patch can be used to operate the implanted device in a number of different ways. For example, each patch could include a fixed program dictating continuous and constant operating parameters for delivery of fluid at a constant rate, wherein each patch corresponds to a different delivery rate. Similarly, each patch could provide operating parameters for the pulsed delivery of fluid, with the implanted device operating at zero fluid delivery for periods of time, and then pulsing a predetermined volume of fluid as needed. Also, the fixed program could include operating instructions for altering the operating parameters of the device at a predetermined schedule so that the operating parameters change as a function of time. Any number of similar programs could be included as fixed programs for the patches of the present invention, with each fixed program being specific to its corresponding patch.

In a preferred embodiment of the system, each patch may additionally include an indicia, wherein the indicia indicates the content of the pre-programmed control program. The indicia may be represented by such things as colors, letters, roman numerals, numbers, shapes, etc.

The above patch and system may be used with a method for providing a control program to an implantable device. A preferred method comprises the steps of associating a pre-programmed patch with an external portion of a patient proximate an implanted medical device, transmitting a control signal from the patch to the implanted device, and altering the operation of the implanted device according to the control signal. If reprogramming instructions are sent from the device, the step of altering the operation comprises the steps of reprogramming the implanted device with the reprogramming instructions, and altering the operation of the device according to the reprogramming instructions. If a control signal is sent either instead of, or in addition to the reprogramming signal, the step of altering the operation may comprise the steps of receiving the power signal from the pre-programmed patch, and altering the operation of the device according to the characteristics of the power signal.

The signal transmitted is preferably sent through an RF signal or though an electric field flux. Preferably, if the step of transmitting comprises the step of transmitting an RF power signal, the step of altering, preferably comprises the step of altering device operation according to at least one of the frequency, amplitude, time, and duration of the power signal.

If, alternatively, the step of transmitting comprises the step of creating a magnetic field through a coil in the pre-programmed patch, wherein the magnetic field extends at least partially to a corresponding coil in the implanted device, then the step of altering may comprise the step of altering device operation according to the flux of the magnetic field through the coil in the implanted device. The magnetic field flux may be altered in a number of ways, through alteration of the density of the field through the coil in the implanted device. Such an alteration may be accomplished by any number of conventional means, including altering the flow rate of current through the coil in the patch.

The present invention is additionally directed to a method for providing replaceable transcutaneous power to an implantable device, comprising the steps of associating an autonomously powered patch with an external portion of a patient proximate an implanted medical device and then transmitting a power signal from the patch to the implanted device so as to provide operative power to same. Preferably, the method additionally comprises the step of replacing the autonomously powered patch with a second autonomously powered patch when the original patch has run out of power.

The patches of the present invention may additionally be used with a method for providing outpatient use of an implanted medical device. The method comprises the steps of providing a patient with at least two self-contained control patches. Each of the patches is configured as described in the system above. Additionally, instructions to the patient on when to apply a patch having a particular indicia are provided by, for example, the physician.

### BRIEF DESCRIPTION OF THE DRAWINGS

FIG. 1 is a perspective view of an autonomous patch of the present invention, with a cut away section of the housing showing, as an example, an RF transmitter within the housing.

FIG. 2 is a perspective view of an autonomous patch of the present invention, with a cutaway section of the housing showing, as an example, coil-to-coil transmitter within the housing;

FIG. 3 is a diagram of coil-to-coil inductive energy transfer showing an approximation of the electric field created by the coil of the present device; and

FIG. 4 is a perspective view of two of the autonomous patches of the present invention, each marked with indicia for use with the patch system disclosed herein.

### DETAILED DESCRIPTION OF THE INVENTION

While this invention is susceptible of embodiment in many different forms, there is shown in the drawings, and will be described in detail, several specific embodiments with the understanding that the present disclosure is to be considered as an exemplification of the principles of the invention and is not intended to limit the invention to the embodiments illustrated.

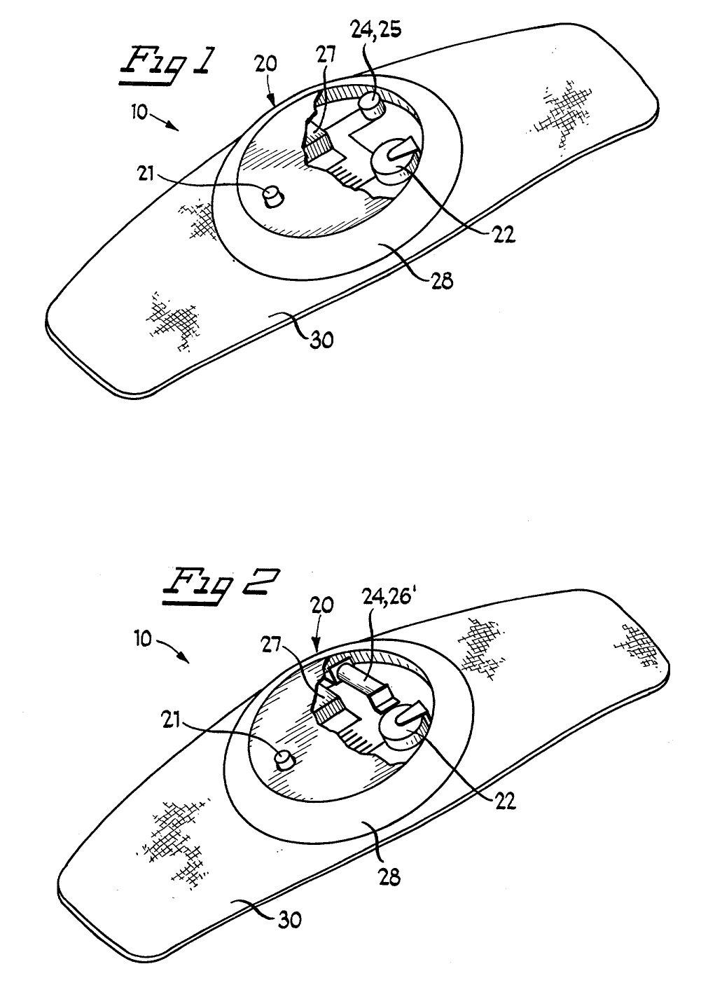

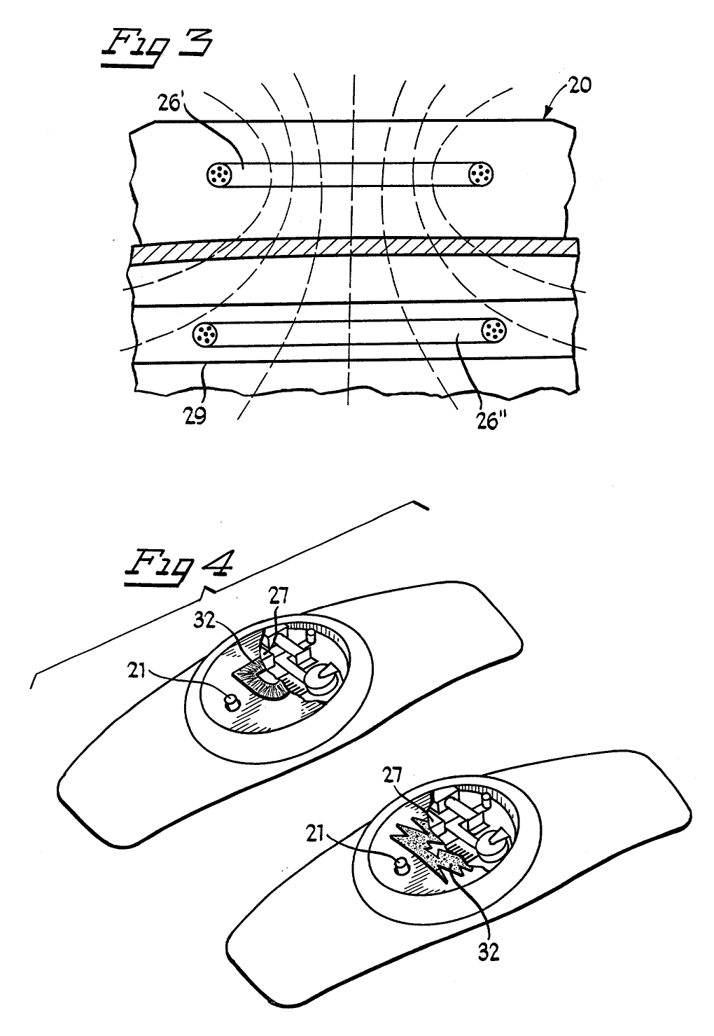

Autonomous patch 10 is shown in FIG. 1 as comprising housing 20, power source 22, transmitting means 24, and storage device 27. Patch 10 is configured for removable placement upon the skin or other tissue of a patient, generally in proximate relationship with an already-implanted medical device 29 (FIG. 3). Once operatively placed upon a patient, patch 10 allows for transcutaneous programming and/or power transfer to the implanted device 29, wherein the programming/power transfer is dictated by a pre-programmed control program contained within storage device 27 of patch 10.

Patch 10 may be used with any number of conventional implanted devices. For example, defibrillators, pacemakers, neurostimulators, cochlear implants and implantable pumping devices are all devices that could be coupled with patch 10. The implantable pumping devices are an especially important category, as the alteration of the operational parameters of implantable pumps can be frequent. Such pumps may include positive displacement pumps, dynamic pumps, lift pumps, electromagnetic pumps, and osmotic pumps, among others. Further, patch 10 may be used with a device or devices intended to be used in conjunction with one of the above devices. For example, patch 10 could alter the operating parameters of an electrically powered valve mechanism associated with an implanted pump.

Generally, each of the above devices may comprise an electrically or electrochemically powered device capable of providing medical treatment. The operational control of which may be alterable via a conventional RF receiver (or other similar type) in association with an external/remote operational control altering means. Such an alteration could include changing the stimulation voltage of a neurostimulator, or the drug delivery flow rate of an implantable pump, for example. Other alterations could include modifying the delivery rate of a valve associated with an implantable pump, or altering a control mechanism for an osmotic pump so as to cause changes in its osmotic delivery rate. In any case, patch 10 allows for at least one of programming and power to be transmitted transcutaneously to the implanted medical device.

Housing 20 of patch 10 is shown in FIG. 1 as encompassing transmitting means 22, power supply 24 and storage device 27 within sealed compartment 28. Housing 20 additionally includes on/off switch 21, for beginning and halting the operation of patch 10. Housing 20 can have any geometrical shape, including, but not limited to hemispherical, cylindrical, cubic, and the like, as may be needed for particular medical applications. As can be seen, housing 20 is associated with attachment means 30, which can include an adhesive bandage (as shown), an adhesive backing on sealed compartment 28, or any number of other means for securing the device, allowing the device to be adhered to and then removed from a particular patient, as needed. For example, attachment means could comprise a bracelet having a hook and eye latch, or Velcro® latch, or an elastic band, as well as other conventional means for attachment.

Housing 20 preferably includes a display device (not shown) associated with the external portion of housing 20. Display device can include any number of conventional displays, including an LCD screen or the like. Display device is associated with the components of patch 10, so that, after activation of the patch 10, any number of operational parameters can be displayed to the user. Such parameters could include the power level of the device, the amount of medicament remaining within the implanted device, the time course of treatment to that time, or any other necessary or desired operating parameter.

Power source 22 is shown in FIG. 1 within compartment 28 as comprising a conventional button cell lithium ion battery which is in electrical communication with all components of patch 10, including transmitting means 24. As will be understood by those having ordinary skill in the art, any type of power source, including other types of batteries, can be used as the power source provided it has the appropriate capacity and energy density to enable operative transmission by transmitting means 24 to implanted device 29 for a desired period of time. It is contemplated that the power source 22 may be replaced as needed by replacing the battery, or by recharging the power source 22 in any conventional means. It is preferred, however, that the patch 10 comprises a single-use power source 22 that is replaced with an additional patch 10 having a fresh power source 22, when needed, as will be discussed below.

Additionally, it is contemplated, though not shown in the drawings, that the compartment 28 additionally includes a releasable plastic strip between power source 22 and the electrical leads for delivering power to patch 10. The strip creates an open circuit state within patch 10 so that, in a storage environment, power source 22 can be inserted and left within compartment 28 without activating patch 10 or draining power source 22. Thereafter, the plastic strip can be removed, placing power source 22 in operative electrical contact with the components of patch 10. Other structures and devices that operate similarly to the plastic strip could alternatively be used.

Transmitting means 24 can comprise any of a number of devices capable of transmitting a signal and/or projecting a field as required by the function of the particular implanted device. For example, transmitting means can comprise RF transmitter 25 for transmitting radio frequency signals. On the other hand, and as shown in FIG. 2, transmitting means 24 could comprise coil 26′, which is capable of producing an electric field upon application of a current. Coil 26′ may be paired with another coil, coil 26″, to create a current in coil 26″ via mutual inductance. An example of mutual inductance is shown in FIG. 3, in which an electric field is projected from coil 26′, and coil 26″ is placed within the field. Once within the field, a current is produced within coil 26″ via inductance, and the current is proportional to the flux of the electric field produced by coil 26′ through coil 26″.

Storage device 27 comprises any number of types of memory-storage apparatuses, including programmable DRAM and SDRAM. Importantly, storage device 27 of the present invention includes one or more pre-programmed control programs that, as will be explained further below, direct the signal sent by transmitting means 24 so as to alter the operation of implanted device as desired. Actual programming can readily be accomplished by those with ordinary skill in the art using conventional microprocessor programming techniques.

The pre-programmed control program of patch 10 comprises a fixed program. That is to say, once patch 10 has been assembled, and storage device 27 has been programmed with the control program, the patch 10 is in final condition. The control program that is associated with the patch 10 will not be changed or altered during the operation of the device. Instead, it will remain fixed, providing the same control signals to the implanted device throughout its operation.

In operation, patch 10 is placed onto the skin of a patient using attachment means 30 at or near the proximate location of an implanted medical device. Patch 10 includes at least enough power in power source 22 to operate transmitting means 24 and storage device 27 throughout its operative life. Once in place, patch 10 can be used to transmit at least one of power or programming to the implanted device.

In one embodiment of the present invention, patch 10 transmits solely power to the implanted device. In this embodiment, the power is transmitted via transmission means 24, described above. For example, in the embodiment of the present invention shown in FIG. 1, transmission comprises an RF transmitter 25 for transmitting power via a radio frequency to implanted device. Alternatively, transmission means 24 could also comprise another transmission method, such as is shown in FIG. 2 with coil 26′ creating an electric field that creates an electric flux in coil 26″, transmitting power from one coil to the other.

When power is transmitted from patch 10 to the implanted device, the power can be used in a number of ways. The implanted device could simply use the transmitted power to operate under standard operating conditions. In this case, patch 10 transmits power using, for example, RF transmitter 25, to the implanted device. Implanted device 10 receives the power, and continues to operate as normal. The characteristics of the power signal transmitted from patch 10 do not effect operation of the implanted device at all, but instead simply act as a remote power source. Advantageously, when patch 10 depletes the installed power source 22, a new patch can be placed on the patient, without lengthy interruption of the operation of the device or requiring time-consuming recharging.

Alternatively, the characteristics of the signal transmitted from transmission means 24 could be used to direct the operation of the implanted device. For example, storage device 27 of patch 10 can contain a specific program for altering the frequency and/or amplitude of an RF signal sent from RF transmitter 25, or for altering the electric field produced by coil 26′. The alterations in the power signal can be used to directly manipulate the implanted device, providing additional power or removing power as needed. For example, an implanted pharmaceutical pump can be manipulated to increase the delivery rate of fluid by increasing the power to the pump. Thus, the simple manipulation of transmitted power can allow a user to control an implanted device.

In another embodiment, patch 10 can be used to only transmit a fixed control signal to an implanted device that is operating under its own power. In this embodiment, transmitting means 24 (preferably an RF transmitter 25) sends a signal to the implanted device, the characteristics of which are regulated by the storage device 27. The implanted device receives the signal, and uses that signal to reprogram the implanted device as desired. Such a signal can be used to modify voltages of the device, fluid delivery rates, sensitivities of sensors, etc. Essentially, any programmable commands may be transmitted via transmitting means 24, as would be known by one of ordinary skill in the art.

Preferably, patch 10 is capable of combining both of the functions discussed above. That is, patch 10 is preferably capable of transmitting both power and fixed control signals to the implanted device.

In one preferred embodiment of the present invention the patch device disclosed above is used with one or more additional patch devices in a medical treatment kit, shown in FIG. 4. In this embodiment, storage device 26 of each patch is preprogrammed with a specific program for transfer of instructions or power to the implanted medical device. For example, in the embodiment discussed above wherein the implanted medical device comprises a pharmaceutical pump, the individual patches may contain specific and different reprogramming information for delivery of a drug at varying delivery rates. Further, the individual patches may additionally transfer a lesser or greater magnitude of power to the implanted device, thereby facilitating the varying delivery rates. Each patch, however, corresponds to discrete sets of programming signals and/or power transfer patterns so that the placement of a particular patch in operable position on a patient facilitates the particular treatment regimen or regimens associated with that particular patch.

Preferably, within this system, each patch is marked on its external side with indicia 32, as can be seen in FIG. 4. Indicia 32 may include numbers, letters, roman numerals, colors, shapes, and the like, with each indicia corresponding to the particular control program, or power level, of the particular patches in the patch kit. As will be explained further below, the relationship of the indicia 32 with the particular delivery/power program associated with each patch helps to facilitate easy and reliable reprogramming (or continued operation) of the implanted devices, as needed.

The present system would be advantageous, for example, in an outpatient pain treatment regimen. After implanting a pharmaceutical pump containing a pain medication such as morphine, a physician could issue one or more patches containing delivery instructions and/or power for the implanted device. The doctor could then issue specific patches to the patient, with each patch corresponding with a specific drug delivery rate. Included with these patches would be instructions for their use, such as, for example, that patch “A” can be used for light pain, patch “B” for increased pain, and patch “C” for severe pain. The patient could, on their own initiative, or at the direction of a medical caregiver, alter the delivery rates of the implanted pharmaceutical pump simply by removing one patch, and replacing it with another. In this way, the physician may allow a patient the ability to modify the dose delivered from a pump within limits specified by the physician. The control programs contained within the patches given to the patient determine the limits of the treatment regimen.

The present medical treatment kit could similarly be used with numerous other applications, as would be known by one of ordinary skill in the art. For example, the patches could be used to adjust the frequency within a neurostimulator for treatment of pain and/or tremors, or for adjusting the frequency/cadence of a pacemaker. Of course, numerous other applications could also be envisioned for the teachings of the present invention.

The foregoing description merely explains and illustrates the invention and the invention is not limited thereto except insofar as the appended claims are so limited, as those skilled in the art who have the disclosure before them will be able to make modifications without departing from the scope of the invention.

What is claimed is:

1. A self contained control patch for an implanted medical device, comprising:
a housing capable of being removably associated with an external surface of a patient, wherein the housing includes:
means for transmitting at least one of a transcutaneous power signal and a transcutaneous control signal to an implanted device;
a pre-programmed storage device containing at least one fixed control program, wherein the fixed control program dictates the nature of the signal from the transmitting means; and
a power source for providing operational power to at least one of the transmitting means and the storage device.

2. The patch according to claim 1, wherein the transmitting means comprises an RF transmitter, wherein the fixed control program dictates at least one of the frequency and amplitude of the RF signal.

3. The patch according to claim 1, wherein the transmitting means comprises a conductive coil, the conductive coil being coupled with a conductive coil associated with the implanted device so as to allow the inductive transfer of power from the transmitting means to the implanted device, the fixed control program dictating the flux of the electric field through the coil in the implanted device.

4. The patch according to claim 1, wherein the transmitting means comprises means for transmitting only a power signal to an implanted device.

5. The patch according to claim 1, wherein the transmitting means comprises means for transmitting only a control signal to an implanted device.

6. The patch according to claim 1, wherein the transmitting means comprises means for transmitting both a transcutaneous power signal and a transcutaneous control signal to an implanted device.

7 The patch according to claim 1, additionally comprising means for attaching the housing of patch to a patient.

8. The patch according to claim 7, the attachment means comprising an adhesive associated with at least a portion of the housing.

9 The patch according to claim 7, the attachment means comprising a bandage material having an adhesive thereon, the housing being associated with the bandage.

10. The patch according to claim 7, the attachment means comprising a bracelet associated with the housing.

11. A pre-programmed treatment system, comprising:
at least two self-contained control patches, each patch comprising:
a housing capable of being removably associated with an external surface of a patient;
means for transmitting at least one of a transcutaneous power signal and a transcutaneous control signal to an implanted device;
a pre-programmed storage device containing at least one fixed control program, wherein the fixed control program dictates the nature of the signal from the transmitting means; and
a power source for providing operational power to at least one of the transmitting means and the storage device.
wherein each control patch contains a separate fixed preprogrammed control program.

12. The system according to claim 11, wherein transmitting means of each patch comprises an RF transmitter, the fixed control program dictating at least one of the frequency and amplitude of the RF signal.

13. The system according to claim 11, wherein the transmitting means of each patch comprises a conductive coil, the conductive coil being coupled with a conductive coil associated with the implanted device so as to allow the inductive transfer of power from the transmitting means to the implanted device, the fixed control program dictating the flux of the electric field through the coil in the implanted device.

14. The system according to claim 11, wherein the transmitting means of each patch comprises means for transmitting only a power signal to an implanted medical device.

15. The system according to claim 11, wherein the transmitting means of each patch comprises means for transmitting only a control signal to an implanted medical device.

16. The system according to claim 11, wherein the transmitting means of each patch comprises means for transmitting both a transcutaneous power signal and a transcutaneous control signal to an implanted device.

17. The system according to claim 11, wherein each patch additionally includes an indicia, wherein the indicia indicates the content of the pre-programmed control program.

18. The system according to claim 17, wherein the indicia comprise one of the group consisting of colors, letters, roman numerals, numbers, and shapes.

19. A method for providing a control program to an implantable device, comprising the steps of:
associating a pre-programmed patch with an external portion of a patient proximate an implanted medical device;
transmitting a fixed control signal from the patch to the implanted device; and
altering the operation of the implanted device according to the fixed control signal.

20. The method according to claim 19, additionally comprises the step of programming a pre-programmed patch with a fixed, program before the step of associating.

21. The method according to claim 19, wherein the step of transmitting a fixed control signal comprises the step of transmitting a control signal containing reprogramming instructions, the step of altering the operation comprising the steps of:
reprogramming the implanted device with the reprogramming instructions; and
altering the operation of the device according to the reprogramming instructions.

22. The method according to claim 19, wherein the step of transmitting a control signal comprises the step of transmitting a fixed control signal containing a power signal, the step of altering the operation comprising the steps of:
receiving the power signal from the pre-programmed patch;
altering the operation of the device according to the characteristics of the power signal.

23. The method according to claim 22, wherein the step of transmitting comprises the step of transmitting an RF power signal, and the step of altering comprises the step of altering device operation according to at least one of the frequency and amplitude of the power signal.

24. The method according to claim 22, wherein the step of transmitting comprises the step of creating a magnetic field through a coil in the pre-programmed patch, wherein the magnetic field extends at least partially to a corresponding coil in the implanted device, the step of altering comprising the step of altering device operation according to the flux of the magnetic field through the coil in the implanted device.

25. A method for providing replaceable transcutaneous power to an implantable device, comprising the steps of:
associating an autonomously powered patch with an external portion of a patient proximate an implanted medical device; and
transmitting a power signal from the patch to the implanted device so as to provide operative power to same.

26. The method according to claim 25, additionally comprising the step of replacing the autonomously powered patch with a second autonomously powered patch when the original patch has run out of power.

27. A method for providing outpatient programming of an implanted medical device, comprising the steps of:
providing a patient with at least two self-contained control patches, each patch comprising:
a housing capable of being removably associated with an external surface of a patient;
means for transmitting at least one of a transcutaneous power signal and a transcutaneous control signal to an implanted device;
a pre-programmed storage device containing at least one fixed control program, wherein the fixed control program dictates the nature of the signal from the transmitting means; and
a power source for providing operational power to at least one of the transmitting means and the storage device;
wherein each patch contains a separate fixed preprogrammed control program, the specific fixed control program being indicated on the patch by a specific indicia, and wherein further each control program may be individually implemented by the placement of the associated patch in operative position upon a patient; and
providing instructions to the patient on when to apply a patch having a particular indicia.
